(19) United States
(12) Patent Application Publication
(10) Pub. No.: US 2026/0151068 A1
(43) Pub. Date: Jun. 4, 2026

(54) OPTIMIZATION OF THE SPATIAL ARRANGEMENT OF ELECTROPHYSIOLOGICAL SENSORS

(71) Applicant: Synchron Australia Pty Limited, Dove, DE (US)

(72) Inventor: Jerel MUELLER, Dover, DE (US)

(73) Assignee: Synchron Australia Pty Limited, Dove, DE (US)

(21) Appl. No.: 19/296,754

(22) Filed: Aug. 11, 2025

Related U.S. Application Data
(60) Provisional application No. 63/727,348, filed on Dec. 3, 2024.

(51) Int. Cl. A61B 5/279; A61B 5/00; A61N 1/05

(52) U.S. Cl. CPC A61B 5/279; A61B 5/4064; A61B 5/407; A61B 5/4082; A61B 5/4839; A61B 5/6868; A61B 5/7225; A61N 1/0529

(57) ABSTRACT

Disclosed here are devices, methods and systems for sensing electrical activity from one or more neural regions of a subject is disclosed, the method comprising: advancing a device to or through a blood vessel in the subject, where the device comprises an electrode array having a plurality of electrodes; detecting an electrical signal of a subject via the plurality of electrodes, where the plurality of electrodes are spaced apart on the electrode array such that the plurality of electrodes are each configured to record distinct electrical signals from each of the one or more neural regions; and analysing the distinct electrical signals using a control unit.

### CROSS-REFERENCE TO RELATED APPLICATION

This application claims the benefit of U.S. Provisional Application No. 63/727,348, filed Dec. 3, 2024, the content of which is incorporated herein by reference in its entirety.

### TECHNICAL FIELD OF THE INVENTION

The present invention relates to a medical device for recording distinct electrical signals from neural regions or for providing stimulation to distinct neural regions.

### BACKGROUND OF THE INVENTION

Any discussion of document, devices, acts or knowledge in this specification is included to explain the context of the invention. It should not be taken as an admission that any of the material forms a part of the prior art base or the common general knowledge in the relevant art in Australia or elsewhere on or before the priority date of the disclosure and broad consistory statements herein.

In the United States alone, nearly two million people suffer from various neuromuscular disorders where control of limbs is severely impaired. In many of these patients, however, the portion of the brain responsible for movement remains intact, and it is disease and trauma to the spinal cord, nerves and muscles that limit mobility, function and independence. For these people, the ability to restore lost control at even a rudimentary level could lead to a greatly improved quality of life. Some approaches for restoring function comprise recording devices that detect, extract, and analyze electrical signals from the implantation site.

The design of electrophysiological sensing/recording devices is challenging and often requires extensive expert evaluation and multiple iterations. In the case of an intravascular recording device, one challenge is designing a device that is sufficiently small to fit within a blood vessel and also sufficiently flexible to traverse and sustain the vascular pathway to the point of deployment. Other challenges include designing a recording device in a manner that captures multiple signals, each of which is robust, and each of which that can be uniquely allocated to multiple channels, thereby supporting a brain computer interface system with multiple degrees of freedom.

Recording devices with too many electrodes can be difficult to manufacture and provides for possible redundant information resulting from overlap of recorded regions in the target area, often called an “oversampling” approach. Recording devices with too few electrodes can be overly dependent on accurate placement of the device as well as dependent on long term viability of the electrodes.

Thus, there remains a need to design an intravascular recording device with a number of electrodes that has sufficient redundancy (i.e., is not dependent on only a few electrodes) and that are spaced in a manner to maximize neural recording from regions of the brain. The regions of the brain are each characterized by distinctly different electrical signatures and characteristics such that different electrical patterns can be recorded from each of them respectively, thereby supporting different channels.

### SUMMARY OF THE INVENTION

In some aspects, the techniques described herein relate to a method of sensing electrical activity from one or more neural regions of a subject, the method including:
advancing a device to or through a blood vessel in the subject, where the device includes an electrode array having a plurality of electrodes; sensing electrical activity via the plurality of electrodes; detecting an electrical signal of a subject, where the plurality of electrodes are spaced apart on the electrode array such that the plurality of electrodes are each configured to record distinct electrical signals from each of the one or more neural regions, wherein the plurality of electrodes are spaced apart such that a resulting coverage field of each of the plurality of electrodes minimizes overlap between each of the plurality of electrodes; and
analysing the distinct electrical signals using a control unit.

In some aspects, the techniques described herein relate to a method, further including deploying the electrode array after advancing the device to or through the blood vessel, where deployment of the electrode array brings each of the plurality of electrodes into engagement with a wall of the blood vessel.

In some aspects, the techniques described herein relate to a method, wherein the device is an intravascular device deployed within the blood vessel.

In some aspects, the techniques described herein relate to a method, wherein the device is a transvascular device.

In some aspects, the techniques described herein relate to a method, wherein deploying the electrode array further includes deploying the electrode array to the subarachnoid space.

In some aspects, the techniques described herein relate to a method, wherein deploying the electrode array further includes deploying the electrode array to the cortical surface.

In some aspects, the techniques described herein relate to a method, wherein deploying the electrode array further includes deploying the electrode array within the cortical tissue.

In some aspects, the techniques described herein relate to a method, wherein the plurality of electrodes record distinct electrical signals from one or more neural regions at the spinal cord of the subject.

In some aspects, the techniques described herein relate to a method, wherein the device is an intravascular device deployed within a blood vessel of the spinal cord.

In some aspects, the techniques described herein relate to a method, wherein the device is a transvascular device.

In some aspects, the techniques described herein relate to a method, wherein deploying the electrode array further includes deploying the electrode array to the epidural space.

In some aspects, the techniques described herein relate to a method, wherein deploying the electrode array further includes deploying the electrode array to the subarachnoid space.

In some aspects, the techniques described herein relate to a method, wherein deploying the electrode array further includes deploying the electrode array to the spinal cord surface.

In some aspects, the techniques described herein relate to a method, wherein deploying the electrode array further includes deploying the electrode array within the spinal cord.

In some aspects, the techniques described herein relate to a method, further including extracting the distinct electrical signals via the control unit and distilling information from the distinct electrical signals to generate one or more control signals sending specific instructions to the device.

In some aspects, the techniques described herein relate to a method, wherein extracting the distinct electrical signals includes temporally filtering the distinct electrical signals.

In some aspects, the techniques described herein relate to a method, wherein extracting the distinct electrical signals includes spatially filtering the distinct electrical signals.

In some aspects, the techniques described herein relate to a method, wherein extracting the distinct electrical signals includes thresholding the distinct electrical signals.

In some aspects, the techniques described herein relate to a method, wherein two or more of the plurality of electrodes are configured to record distinct electrical signals from each of the one or more neural regions.

In some aspects, the techniques described herein relate to a method of sensing electrical activity from one or more neural regions, the method including: advancing a device through the subarachnoid space in a subject, where the device includes an electrode array having a plurality of electrodes; detecting an electrical signal of a subject, where the plurality of electrodes are spaced apart on the electrode array such that the plurality of electrodes are each configured to record distinct electrical signals from each of the one or more neural regions; and analysing the distinct electrical signals using a control unit, wherein the distinct electrical signals each correspond to distinct recordings of electrical activity within the subject.

In some aspects, the techniques described herein relate to a method, further including deploying the electrode array within the subarachnoid space adjacent to a surface of the brain, wherein deployment of the electrode array brings each of the plurality of electrodes into engagement with the surface of the brain.

In some aspects, the techniques described herein relate to a method, wherein the plurality of electrodes include one or more pairs of electrodes, wherein each pair of electrodes is configured to record distinct electrical signals from each of the one or more neural regions.

In some aspects, the techniques described herein relate to a method, wherein the plurality of electrodes include one or more sets of three electrodes, wherein each set of three electrodes is configured to record distinct electrical signals from each of the one or more neural regions.

In some aspects, the techniques described herein relate to a method, wherein the plurality of electrodes include one or more sets of two or more electrodes, wherein each set of two or more electrodes is configured to record distinct electrical signals from each of the one or more neural regions.

In some aspects, the techniques described herein relate to a method, further including extracting the distinct electrical signals via the control unit and distilling information from the distinct electrical signals to generate one or more control signals sending specific instructions to the device.

In some aspects, the techniques described herein relate to a method, wherein extracting the distinct electrical signals includes temporally filtering the distinct electrical signals.

In some aspects, the techniques described herein relate to a method, wherein extracting the distinct electrical signals includes spatially filtering the distinct electrical signals.

In some aspects, the techniques described herein relate to a method, wherein extracting the distinct electrical signals includes thresholding the distinct electrical signals.

In some aspects, the techniques described herein relate to a method, wherein two or more of the plurality of electrodes are configured to record distinct electrical signals from each of the one or more neural regions.

In some aspects, the techniques described herein relate to a method of providing stimulation to one or more neural regions of a subject, the method including: advancing a device to or through a blood vessel in the subject, where the device includes an electrode array having a plurality of electrodes; stimulating the one or more neural regions via the plurality of electrodes; where the plurality of electrodes are spaced apart on the electrode array such that the plurality of electrodes are each configured to stimulate one or more distinct neural regions, wherein the plurality of electrodes are spaced apart such that a resulting stimulation field of each of the plurality of electrodes minimizes overlap between each of the plurality of electrodes, wherein stimulation is delivered using a control unit.

In some aspects, the techniques described herein relate to a system for sensing electrical activity from one or more neural regions of a subject, the system including: an electrode array including a plurality of struts, where the electrode array is configured to transition between a constrained configuration and a deployed configuration in which the electrode array is implanted within a blood vessel; a plurality of electrodes spaced apart on the electrode array such that the plurality of electrodes are each configured to record distinct electrical signals from each of the one or more neural regions, wherein the plurality of electrodes are spaced along a longitudinal axis of the electrode array, wherein the plurality of electrodes are each configured to sense distinct electrical activity from distinct neural regions within the blood vessel; and a control unit configured to receive and analyze the distinct electrical signals, wherein the distinct electrical signals each correspond to distinct recordings of electrical activity within the subject.

In some aspects, the techniques described herein relate to a system, wherein the plurality of electrodes are offset from each other in a plane transverse to the longitudinal axis of the electrode array.

In some aspects, the techniques described herein relate to a system, wherein a diameter of the electrode array is 10.5 mm.

In some aspects, the techniques described herein relate to a system, wherein the electrode array includes a cylindrical shape in the deployed configuration.

In some aspects, the techniques described herein relate to a system, wherein the plurality of electrodes are spaced in rows of electrodes.

In some aspects, the techniques described herein relate to a system, further including a plurality of electrical vias each connected to each of the plurality of electrodes, wherein the plurality of electrical vias is coupled to a proximal end of the electrode array and connects to a lead and a control unit of the system.

In some aspects, the techniques described herein relate to a system, wherein each of the plurality of electrical vias are routed so as to not cross another electrical via.

In some aspects, the techniques described herein relate to a system, wherein the plurality of electrodes include a substance configured to be released by the plurality of electrodes.

In some aspects, the techniques described herein relate to a system, wherein the substance is a therapeutic drug.

In one variation, a method of sensing electrical activity from one or more neural regions of a subject is disclosed, the method comprising: advancing a device to or through a blood vessel in the subject, where the device comprises a frame structure having a plurality of electrodes; applying energy via the plurality of electrodes; detecting an electrical signal of a subject, where the plurality of electrodes are spaced apart on the frame structure such that the plurality of electrodes are each configured to record distinct electrical signals from each of the one or more neural regions, wherein the plurality of electrodes are spaced apart such that the resulting field of each of the plurality of electrodes minimizes overlap between each of the plurality of electrodes; and analysing the distinct electrical signals using a control unit.

In some variations, the method can further comprise deploying the frame structure after advancing the device to or through the blood vessel, where deployment of the frame structure brings each of the plurality of electrodes into engagement with a wall of the blood vessel.

In some variations, the device can be an intravascular device deployed within the blood vessel.

In some variations, the device can be a transvascular device. Deploying the frame structure can further comprise deploying to the subarachnoid space, the cortical surface, or to or within the cortical tissue.

In some variations, the plurality of electrodes can record distinct electrical signals from one or more neural regions at the spinal cord of the subject.

In some variations, the device can be an intravascular device deployed within a blood vessel of the spinal cord.

In some variations, the device can be a transvascular device deployed to the epidural space, the subarachnoid space, the spinal cord surface, or within the spinal cord.

In some variations, the method can further comprise extracting the distinct electrical signals via the control unit and distilling information from the distinct electrical signals to generate one or more control signals sending specific instructions to the device.

In some variations, the method can further comprise signal processing to temporally filtering the distinct electrical signals, spatially filtering the distinct electrical signals, or threshold the distinct electrical signals.

In some variations, two or more of the plurality of electrodes can be configured to record distinct electrical signals from each of the one or more neural regions.

In other variations, a system for sensing electrical activity from one or more neural regions of a subject is disclosed, the system comprising: a frame structure comprising a plurality of struts, where the frame structure is configured to transition between a constrained configuration and a deployed configuration in which the frame structure is implanted within a blood vessel; a plurality of electrodes spaced apart on the frame structure such that the plurality of electrodes are each configured to record distinct electrical signals from each of the one or more neural regions, wherein the plurality of electrodes are spaced along a longitudinal axis of the frame structure, wherein the plurality of electrodes are each configured to sense distinct electrical activity from distinct neural regions within the blood vessel; and a control unit configured to receive and analyze the distinct electrical signals, wherein the distinct electrical signals each correspond to distinct recordings of electrical activity within the subject.

In some variations, the plurality of electrodes can be offset from each other in a plane transverse to the longitudinal axis of the frame structure.

In some variations, a diameter of the frame structure can be 10 mm.

In some variations, the frame structure can comprise a cylindrical shape in the deployed configuration.

In some variations, the plurality of electrodes can be spaced in rows of electrodes.

In some variations, the system can further comprise a plurality of electrical vias each connected to each of the plurality of electrodes, wherein the electrical via is coupled to a proximal end of the frame structure and connects to a medical lead and a control unit of the system. In some variations, each of the plurality of electrical vias can be routed so as to not cross another electrical via.

### BRIEF DESCRIPTION OF THE DRAWINGS

Variations of the present invention are hereafter described, by way of non-limiting example only, with reference to the accompanying drawing. Like reference numerals in the drawings indicate identical or functionally similar features/elements throughout. All dimensions shown in the drawings are exemplary.

FIG. 1 is a diagrammatic illustration of a system for controlling use of an apparatus coupled to an animal or a human.

FIG. 2A is a diagrammatic illustration showing parts of the system shown in FIG. 1.

FIG. 2B is a diagrammatic illustration showing an additional variation of the system comprising two or more stents.

FIG. 3 is a diagrammatic illustration showing parts of the system shown in FIG. 1.

FIG. 4 is a diagrammatic illustration of a control unit of the system shown in FIG. 1.

FIG. 5A illustrates one variation of an electrode array with spaced electrodes.

FIG. 5B illustrates a diagram of electrode coverage of the electrode array of FIG. 5A.

FIG. 6 illustrates an electrode array having one or more electrical vias each connecting to an electrode.

FIGS. 7A and 7B illustrate one variation of an electrode configuration and its corresponding electrode coverage diagram.

FIGS. 8A to 8F illustrate electrode coverage diagrams of the electrode array of FIG. 5A for various cylindrical diameters.

FIG. 9 shows diagrammatic illustrations of different electrode configurations.

FIG. 10 shows diagrammatic illustrations of different electrode configurations.

FIG. 11 is a diagrammatic illustration of a medical device of the system shown in FIG. 1.

FIG. 12 shows diagrammatic illustrations of different electrode configurations.

FIG. 13A is a diagrammatic illustration showing wire attachments to an electrode.

FIG. 13B is a diagrammatic illustration showing electrode lead wires wrapped around a shaft and covered in insulation forming a wire bundle or cable.

FIGS. 13C and 13D illustrate variations of leads coupled to a device and, which are configured for repositioning of the lead or device.

FIGS. 14 to 16 are diagrammatic illustrations of various connections to the control unit of the system shown in FIG. 1.

FIGS. 17 and 18 are diagrammatic illustrations showing different stages of deployment of the device.

FIG. 19 is a diagrammatic illustration of a wireless electrode system.

FIG. 20 is a diagrammatic illustration of the system being used to record neural information or stimulation of neurons from the superior sagittal sinus (SSS) or branching cortical veins of a patient using the device.

FIG. 21 shows an image reconstruction of a human brain (eyes facing left) demonstrating superior sagittal sinus and branching cortical veins near the motor cortex (red) and sensory cortex (yellow).

FIG. 22 is a diagrammatic illustration showing a method for stimulation and recording neural information or stimulation of neurons from the visual cortex of a patient using the device.

FIG. 23 is a diagrammatic illustration showing vessels and muscles in a human arm;

FIG. 24 is an illustration of a human hand showing implant location to enable neural stimulation or sensing.

FIG. 25 is a photo of a C-shaped ground electrode.

FIGS. 26A and 26B illustrate one variation of the device having an electrode array with one or more electrodes spaced apart from each other and connected to a lead.

FIGS. 27A and 27B illustrate the threaded tip according to one variation of the invention.

FIGS. 28A to 28D illustrate a lead extender according to one variation of the invention.

### DETAILED DESCRIPTION

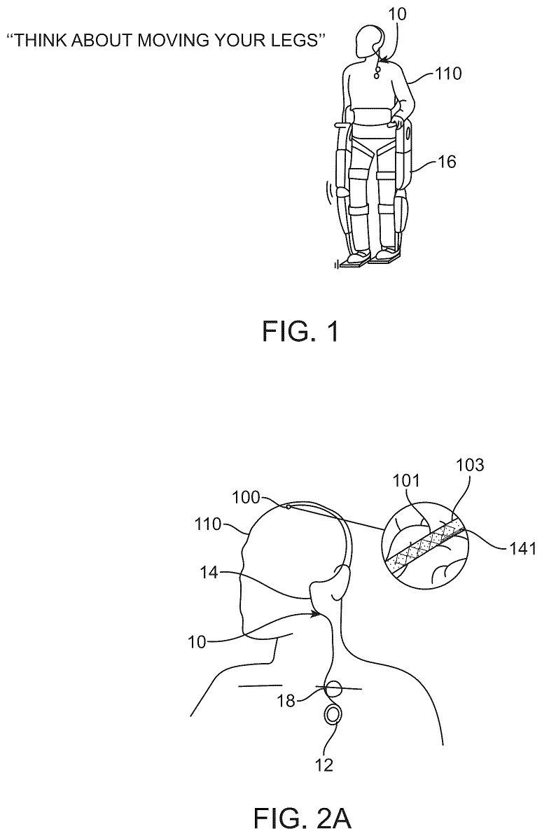

The system 10 shown in FIGS. 1 to 4 includes: 1) a medical device 100 designed for placement within a vessel 103 of a subject 110 to engage the vessel and stimulate and/or sense the activity of media proximal (adjacent or touching) to the device 100, whether this be located inside or outside the vessel 103; 2) a control unit 12 (also referred to as a connector block and telemetry system) adapted for communication with the device; 3) a communication conduit 14 for facilitating communications between the device 100 and the control unit 12; and 4) apparatus 16 coupleable to the animal or human 110, the apparatus 16 adapted for communication with the control unit.

The control unit 12 can be adapted to perform the steps of: (a) receiving data from the device 100 representing activity of media proximal to the device 100; (b) generating control signals for the apparatus 16; and (c) sending the control signals to the apparatus 16. In some variations, the system includes connector block (illustrated by element 12) that functions as connector and acts as an extension of the communication conduit. In variations of the system, the control unit/connector block: is hermetically sealed and insulates the leads from the device to the control unit; can be inserted using zero-contact force attachments or attachments that do not require excessive force to insert (i.e., using balseal spring contacts); has a portion of the lead that is made from a stiffer silicone or similar material for handling and insertion into the connector. Variations of the device can include markers to identify portions of the leads that are stiffer (and can be handled) to distinguish from leads that cannot be handled. Such markers can include line-style markers, different colours or other indicators to clearly identify the regions. Variations of the connector block can have a fitting (e.g., clasp) such that multiple connectors can be inserted (i.e., two contact connectors (with 8 contacts each) for a 16 electrode Stentrode lead). The fitting can ensure securing of the contacts, alignment and prevention of water ingress.

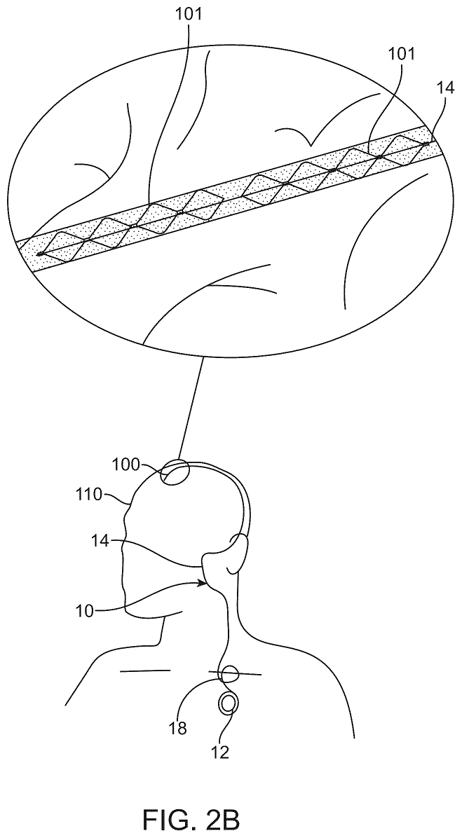

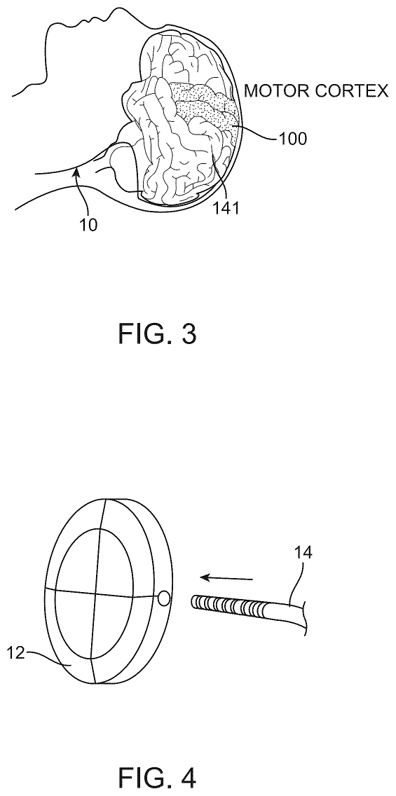

When the medical device 100 is inserted adjacent to the motor cortex in the manner shown in FIGS. 2A, 2B, and 3, the system 10 can be used, for example, to control operation of an exoskeleton, and/or an artificial limb in the manner shown in FIG. 1.

This device 100 is implanted into blood vessels 103, from which, it will utilize electrodes mounted on a self-expanding member 101 to record or stimulate neighboring tissue. Information is to be passed from or to the electrodes through the communication conduit 14, inside of the blood vessel 103, to a telemetry system 12 that, in turn, passes information (using wires or wirelessly) to or from an external apparatus 16, which includes (but is not limited to) one or more of the following:
(a) an exoskeleton; (b) wheelchair; (c) computer; and/or (d) other electrical or electro-mechanical device.

As such, in one specific application, the implanted medical device 100 has the capability to enable a paralyzed patient 110 to use their thoughts directly to command and control a gait aid such as an exoskeleton or robotic legs 16.

Other applications for the implantable medical device 100 include (but are not limited to): (a) detection and prevention of seizures; (b) detection and prevention of involuntary muscular or neural control (for example to alleviate symptoms associated with: (i) multiple sclerosis; (ii) muscular dystrophy; (iii) cerebral palsy; (iv) paralysis and (v) Parkinsons'; (c) detection and therapeutic alleviation of neurological conditions, such as: (i) post-traumatic stress disorder; (ii) obsessive compulsive disorder; (iii) depression; and (iv) obesity; (d) direct brain control of computers and equipment, such as: (i) vehicles; (ii) wheelchairs; (iii) gait aids; robotic limbs; (e) direct input for sensory stimulation for: (i) blindness (connection to a camera); (ii) deafness (connection to microphone); (iiii) proprioception (connection to touch-sensitive robotic and computer systems); (f) internal assessment of personal health and wellbeing: (i) heart rate; (ii) respiration rate; (iii) temperature; (iv) environmental conditions; (v) blood sugar levels; and (vi) other biochemical and neurological markers; (g) internal communication (telepathy) between implanted groups of people utilizing the device for information transmission, auditory, visual and proprioceptive feedback (for example, real time communication of what the implantee sees or hears); and (h) augmentation and optimization of musculoskeletal control and dexterity (for performance enhancement or rehabilitation).

FIG. 2B illustrates a two-stent 101 system. For purposes of illustration, the stents are positioned in a single vessel. However, the stents can be configured such that they can be positioned in separate vessels. Alternatively, the stents can be coupled by one or more wires or conductive elements. Moreover, the system can include active electronics between the stents 101.

The devices described herein can be positioned in any number of areas of brain structures depending upon the desired outcome. For example, as discussed in Teplitzky, Benjamin A., et al. “Computational modeling of an endovascular approach to deep brain stimulation.” Journal of Neural Engineering 11.2 (2014): 026011.stents can be positioned as follows: Internal capsule for depression and obsessive compulsive disorder (OCD); thalamus for epilepsy (E), Parkinsons'Disease, essential tremor, Tourette syndrome, consciousness disorder, chronic pain, obsessive compulsive behavior; fornix for Alzheimer's disease; globus pallidus internus for dystonia, depression, Tourette syndrome; hippocampus for epilepsy; hypothalamus for obesity, anorexia nervosa; inferior thalamic peduncle for depression and obsessive compulsive disorder; lateral habenula for depression, obesity, anorexia nervosa; nucleus accumbens for depression, obsessive compulsive disorder, addiction, obesity, anorexia nervosa; periaqueductal/periventricular for chronic pain; subgenual cingulate white matter for depression; subthalamic nucleus for Parkinson's Disease, dystonia, depression, obsessive compulsive disorder, epilepsy; and ventral capsule for obsessive compulsive disorder.

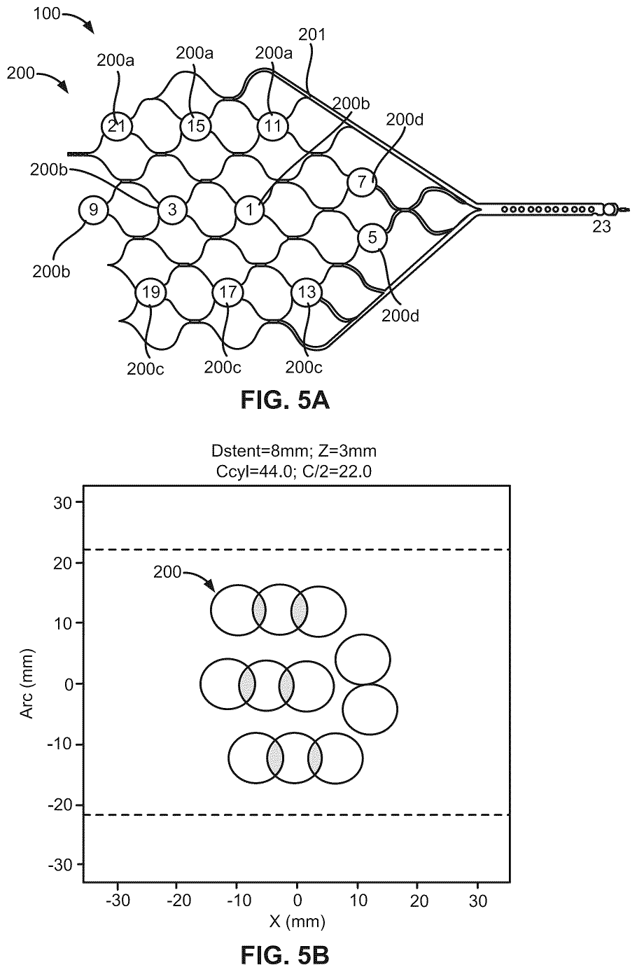

As seen in FIG. 5A, the medical device 100 can comprise an expandable electrode array 201 carrying a plurality of electrodes 200. The electrode array 201 can be expandable so as to bring the plurality of electrodes 200 into engagement with neural regions at a target site.

The plurality of electrodes 200 can be spaced apart such that the plurality of electrodes are each configured to record distinct electrical signals from one or more neural regions in the central nervous system to which the medical device 100 is advanced. Accordingly, the plurality of electrodes 200 can be offset from each other in a plane transverse to the longitudinal axis of the electrode array 201.

As seen in FIG. 5B, the configuration of the plurality of electrodes 200 results in minimal redundancy in order to maximize neural recording from neural regions each characterized by distinctly different electrical signatures or characteristics, such that different electrical patterns can be recorded from each of the neural regions respectively. The spacing of the plurality of electrodes 200 results in a minimum overlap of adjacent electrodes to support separate channel recordings of the neural regions. As a result, each electrode can record separate electrical signals, supporting clear channel recordings.

Referring to FIG. 5B, Z represents the perpendicular offset from the surface of the electrode array (e.g., 3 mm), X represents the longitudinal length along the electrode array in millimeters, and Ccyl represents the circumference of the formed cylinder of the electrode array (e.g., 44.0 mm).

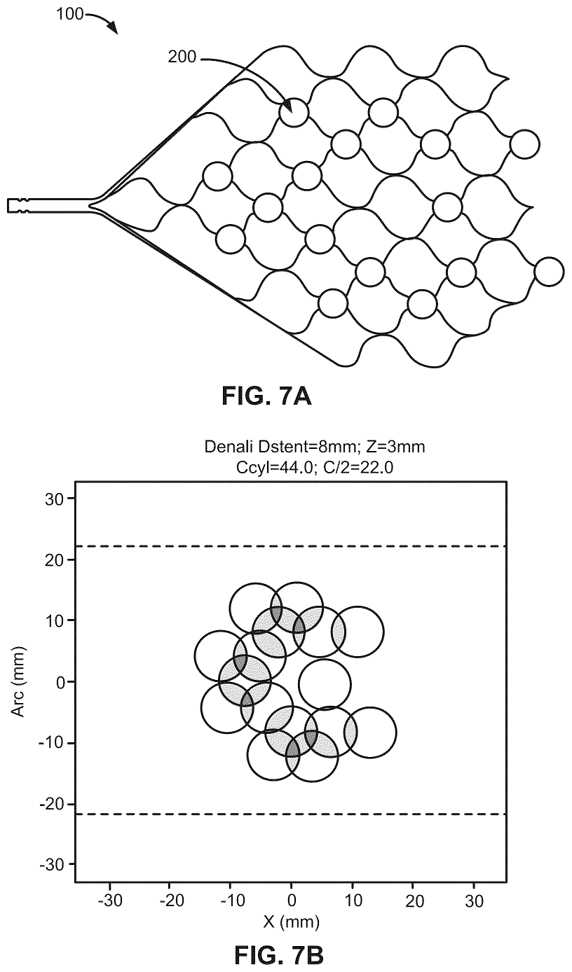

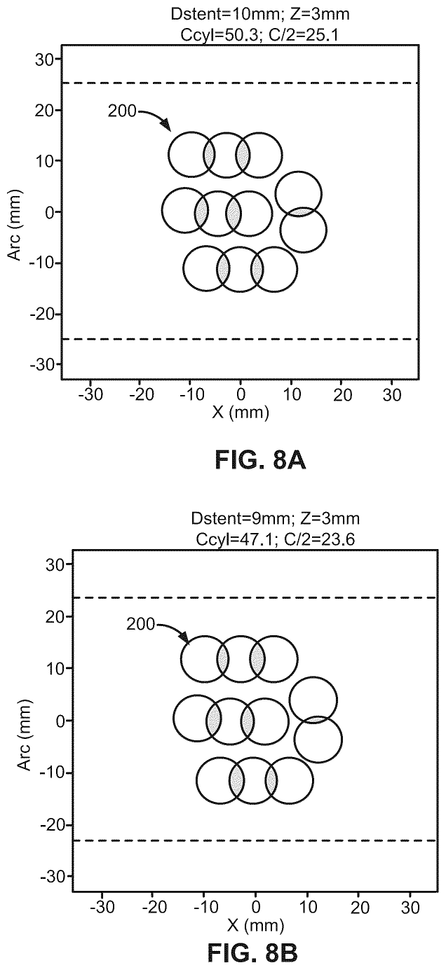

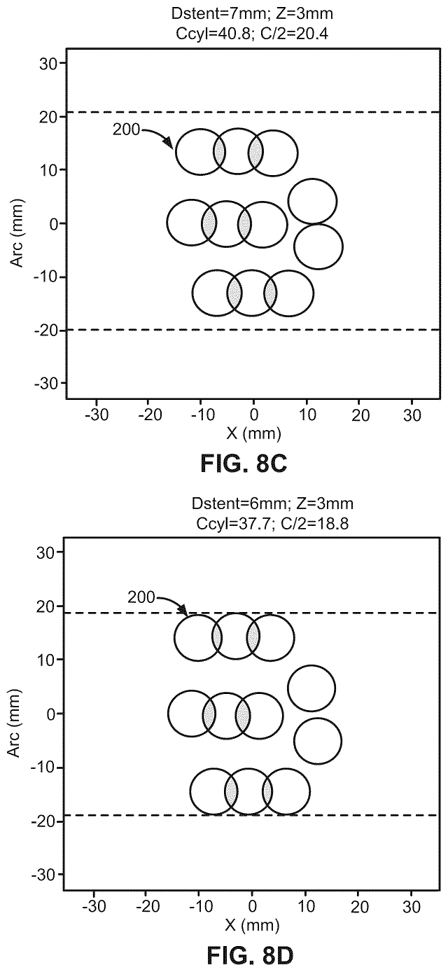

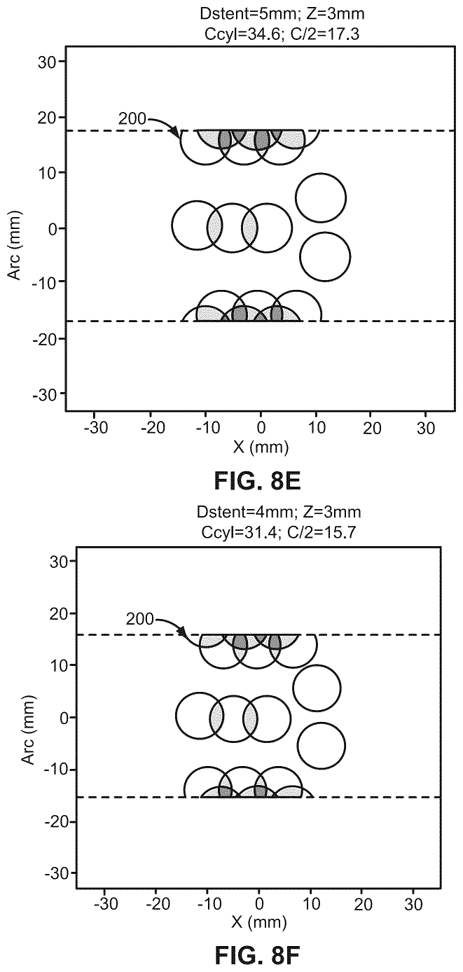

In the diagram shown in FIG. 5B (and FIGS. 7B and 8A to 8F), the space within each individual circle of represent electrode 200 coverage of said electrode. Where two electrodes 200 overlap, the electrode coverage is represented in grey. Where three or more electrodes 200 overlap, the electrode coverage is represented in a darker grey.

Electrode coverage represents a simulation demarcating the region of maximal effects for each of the individual electrodes, defined as greater than or equal to half of the peak effects. For stimulation, the region is the area of voltages greater than or equal to half the peak voltage resulting from stimulation by the associated individual electrode at the offset plane of analysis (e.g.. 3 mm). For recording, the region is the area where the induced voltage on the associated individual electrode by an identical unit of stimulation from each point in the plane of analysis (e.g., 3 mm) is greater than or equal to half the peak voltage induced on the electrode.

In some variations, the device 100 can avoid overlap of stimulation/recording by controlling the timing (i.e., frequency) of stimulation and recording relative to each other. In some variations, the device 100 can avoid overlap of stimulation and recording by the position of the electrodes (i.e., placing electrodes far enough apart to avoid overlap). In some variations, the device 100 can avoid overlap and obscure signal recordings through control of the voltage or current applied to stimulation electrodes (i.e., stimulation amplitude).

Capturing multiple, distinct, robust signals is important in order to uniquely allocate each signal to multiple channels, thereby supporting a brain-computer interface system with multiple degrees of freedom. This is facilitated by placing electrodes such that the resulting receptive field of each electrode minimizes overlap with other, neighboring electrodes. This improves the amount of unique information of the recordings of each distinct channel. In some variations, a minimum viable community of neurons that generate a signal is recorded by each distinct electrode.

The control unit can extract the distinct electrical signals and can distill information from the distinct electrical signals to generate one or more control signals sending specific instructions to the device 100. Extracting the distinct electrical signals can comprise temporally filtering the distinct electrical signals, spatially filtering the distinct electrical signals, thresholding the distinct electrical signals, or a combination thereof. The distinct electrical signals can then be sent to the control unit 12 through the electrical vias and analyzed by the control unit 12 accordingly.

As shown in the top view of the electrode array in FIG. 5A, the plurality of electrodes 200 can be spaced across the electrode array 201 in such a way as to minimize overlap of their respective sensing fields with each other. In this variation, the eleven electrodes 200 can be considered in groups containing electrodes 200a, 200b, 200c, and 200d. The groups comprising electrodes 200a, 200b, and 200c can be separated by rows extending longitudinally with respect to the electrode array. The group comprising electrodes 200d can comprise electrodes closest to the proximal end of the electrode array.

The geometry of the electrode array 201 can have a significant effect on the coverage of each individual electrode. For example, as seen in FIG. 5B, a diameter of about 8 mm results in two instances of electrode overlap between lateral sides of the electrodes in each of electrode 200 groups or rows of electrodes 200 a, b, and c and no overlap between electrodes 200d. Additionally, the placement of the electrodes 200 longitudinally along the frame can influence the overlap of the electrodes within each individual row.

In some variations, the struts have a smaller characteristic length than the electrodes. The characteristic length of the electrodes can be small compared to the distances of the neural structures to being recorded, such that there are only negligible effects by the struts on the electrode coverage.

The shape of the struts of the electrode array can also play an important role in determining the location of the electrodes, and thus their overlap and coverage. For example, struts with larger widths would result in the interaction of the metal struts with the receptive fields of the electrodes becoming more pronounced at closer offsets from the electrode surface.

The size and shape of the electrodes 200 can result in variations of the electrode coverage. For example, larger electrodes can cover a greater region for sensing but can result in overlap between neighboring electrodes.

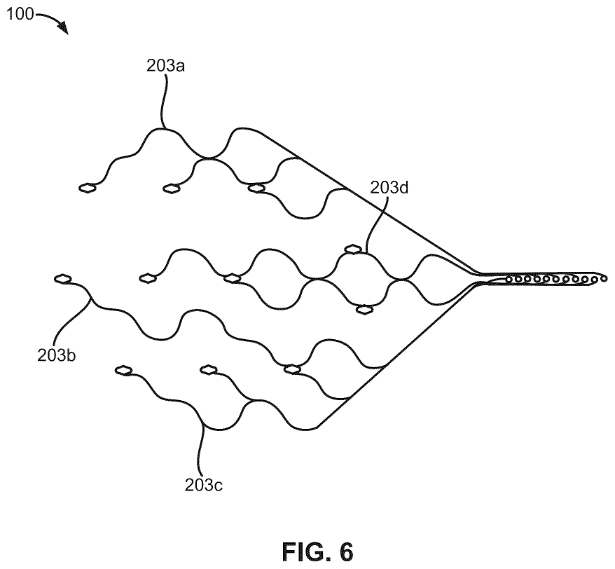

As seen in FIG. 6, each of the electrodes 200 can each be connected to an electrical via 203 leading to the proximal end of the device 100. Each electrical via 203 can be electrically coupled to each electrode 200 accordingly to communicate with the control unit 12. The electrical vias 203 can be connected to a lead that traverses long distances through the blood vessels and to the control unit that digitizes the recorded signals. The electrical vias 203 can be routed such as to not cross any other vias, as well as to ensure that there is a minimum distance to the edge of the struts, neighboring vias, and neighboring electrodes. The electrical vias 203a, b, c, d can each be connected to a single electrode 200a, b, c, d.

In some variations, as seen in FIG. 7A, electrodes 200 can be spaced closer together compared to the electrodes 200 in FIG. 5B. As a result, as seen in FIG. 7B, this results in more overlap in sensing coverage among electrode 200 groups a, b, and c.

FIGS. 8A and 8B illustrate electrode arrays with diameters of about 10 mm and 9 mm, respectively. The configurations in these variations illustrate closer distances between groups of electrodes 200a, b, and c. In these variations, electrodes 200 in group d exhibit some overlap in coverage compared to the electrode array configuration in FIG. 5B.

In other variations, the diameter of the electrode array can be 10.5 mm.

FIGS. 8C, 8D, 8E, and 8F illustrate electrode arrays with diameters of about 7 mm, 6 mm, 5 mm, and 4 mm, respectively. In this configuration, the groups of electrodes 200d do not exhibit some overlap in coverage. However, groups of electrodes 200a and 200c exhibit overlap in electrode coverage. The groups of electrodes 200a and 200c overlapping is a result of the lateral edges of the electrode array overlapping as the diameter decreases. The group of electrodes 200d not overlapping is due to the same mechanism of the diameter decreasing but is a result of the central angle (angle between two radii of a circle) between the electrodes increasing as the diameter of the electrode array 201 decreases. As a result, the projection of the centers of the electrodes 200d on an offset plane are further apart in electrode arrays with smaller diameters.

The distinct electrical signals can be captured by a plurality of electrodes or a single electrode (i.e., electrode-to-signal ratio of 3:2, 2:1, 1:1). In some variations, a plurality of electrical signals can be captured by a single electrode (i.e., electrode-to signal ratio of 1:2, 1:3, 1:4, etc.).

In some variations, the device 100 can be deployed intravascularly within a blood vessel. In other variations, the device 100 can be deployed intravascularly within the spinal cord to target neural regions therein.

In other variations, the device 100 can be deployed transvascularly across the blood vessel. Accordingly, the device 100 can be deployed to the subarachnoid space, the cortical surface, or within the cortical tissue.

In some variations, deployment of the device 100 can be transvascular to target neural regions of the spinal cord. In these variations, the device 100 can be deployed transvascular to the epidural space, subarachnoid space, or at the spinal cord surface. In other variations, the device can be deployed within the spinal cord.

One technical challenge encountered by the inventors is the challenge of creating an electrode array having electrodes spaced apart to efficiently record separate electrical signals, supporting clear multiple channel recordings. One technical solution discovered by the inventors is to set equally spaced electrodes across struts of the electrode array and quantify the sensing spread of each unique electrode shape. The sensing coverage is then scored according to positive metrics (e.g., cumulative full width at half maximum coverage of electrodes, coverage of target neural site) and negative metrics (e.g., overlap of full width at half maximum regions of electrodes, coverage of non-target neural sites). For reference, “full width at half maximum” refers to the distance between the borders at half the maximum of the observed function, often used in engineering to quantifiably determine the region of maximal effects. Multiple evaluations can be performed to account for varying patient anatomy, rotation of the electrode array, or varying depths or locations of the neural tissue to be targeted. After evaluation, a neural sensing score can be output to determine whether the design (e.g., number of electrodes, electrode positions, strut configuration) should be changed or maintained.

Electrode lead wires can be electrically connected to at least one electrode and can be wound around the stent strut lattice such that mechanical compression and extension is not interfered with. Electrode wires may be wound around the stent shaft, thread through a stylet shaft or may form part of the stent shaft directly. Lead wires can form connections with electrode contacts on the opposite end of the stent shaft to the stent, whereby electrical contact a connector block mechanism 12 enables the connection path with external equipment 16, which included but is not limited to computers, wheelchairs, exoskeletons, robotic prosthesis, cameras, vehicles and other electrical stimulation, diagnostic and measurement hardware and software.

The stent 101 can include a plurality of struts coupled together with strut crosslinks. Alternatively, the device 100 includes a stent with any suitable number of electrodes 200 arranged in any suitable configuration. For example, the electrodes can be configured as follows: a sinusoidal arrangement of electrodes, a spiral arrangement of electrodes to enable 360 degree contact of an electrode to the vessel wall once deployed, a reduced amplitude sinusoidal arrangement of electrodes for increased coverage whilst still ensuring only one stent is at each vertical segment; and a dense arrangement of electrodes for increased coverage.

The stent 101 can be laser cut or woven in a manner such that there is additional material or markers where the electrodes 200 are to be placed to assist with attachment of electrodes and uniformity of electrode locations. For example, if a stent 101 was fabricated by laser cutting material away from a cylindrical tube (original form of stent), and, for example, electrodes are to be located at 5 mm intervals on the one axis, then electrode mounting platforms can be created by not cutting these areas from the tube. Similarly, if the stent is made by wire wrapping, then additional material can be welded or attached to the stent wires providing a platform on which to attach the electrodes.

Alternatively, stents can be manufactured using thin-film technology, whereby material (Nitinol and or platinum and or other materials or combinations of) is deposited in specific locations to grow or build a stent structure and/or electrode array

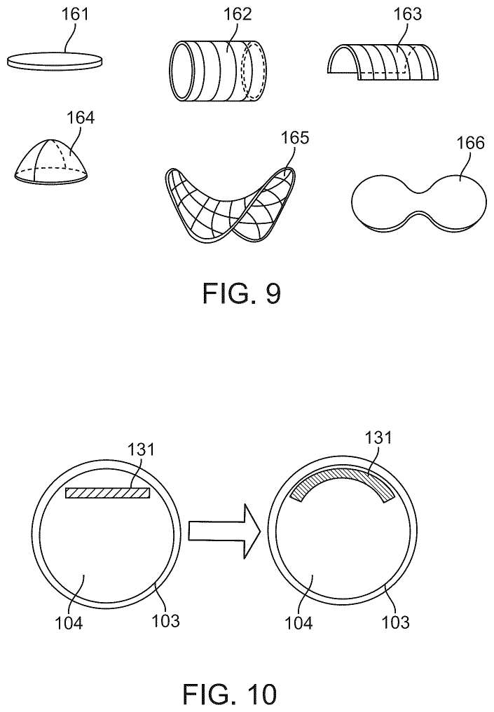

FIG. 9 depicts different electrode geometries which include but are not limited to: flat discs 161; cylinders or rings 162; half-cylinders or rings 163; spheres, domes or hemispheres 164; hyperbolic parabaloids 165; and double electrodes or electrodes whereby they are longer along one axis 166.

As shown in FIG. 10, the electrodes 200 can include shape memory material and hence the electrodes 200 may be uninsulated sections of the device 100. As shown, the electrode 200 inside a patient and the vessel 104 is unobstructed. After activation of shape memory, the electrode 200 conforms to better fit the vessel wall 103.

To enhance contact and functionality of the device 100, electrodes 200 include the attachment of additional material (shape memory alloy or other conducting material) through soldering, welding, chemical deposition and other attachment methods to the stent 101 including but not limited to: directly on or between the stent struts 108; to lead wires 14 passing from the electrodes 200 to wireless telemetry links or circuitry; and directly to an olive 112 placed on the distal aspect of the device 100 to or stent shafts.

To optimise the ability of the electrodes 200 to stimulate or record from medium (including but not limited to neural tissue, vascular tissue, blood, bone, muscle, cerebrospinal fluid), the electrodes 200 may be positioned at pre-determined intervals based on the diameter of the target vessel 103 to allow each of the electrodes 200 to be in contact with the vessel 103 in the same orientation (ie, all electrodes facing to and in contact with the left vessel wall upon deposition). Electrodes 200 may be mounted such that recordings or stimulation can be directed to all 360 degrees of the vessel simultaneously. Similarly, to enhance the recording and stimulation parameters of the electrodes 200, the electrode sizes may be varied, with larger electrodes 200 used to assess greater areas of neighbouring medium with smaller electrodes 200 utilised for localisation specificity.

Alternatively, the electrodes 200 are made from electrically conductive material and attached to one or more stents, which form the device 100 and allow for multiple positions. In this embodiment, the electrodes 200 are made from common electrically active materials such as platinum, platinum-iridium, nickel-cobalt alloys, or gold, and may be attached by soldering, welding, chemical deposition and other attachment methods to one or more lead wires 141, which may be directly attached to the shape memory shaft(s). The electrodes 200 can be one or more exposed sections on the insulated lead wire 141 and the electrode lead wires may be wrapped around one or more shape memory backbones. There may be one or more electrodes and lead wires wrapped around a single shape memory backbone, and, where multiple shape memory backbones are used in the one device, the backbones may have different initial insertion and secondary deposition positions. Thus, they may be used for targeting multiple vessels simultaneously.

As shown in FIG. 12, the electrodes 200 can be designed such that they are carriers of substances 134 and solutions such as therapeutic drugs, including but not limited to anti-thrombogenic, and materials. In this embodiment, the electrodes 200 are designed to release the drugs, either passively through diffusion or through control by an implanted electrical clock or manually through electrical stimulation of the electrodes 200. In this embodiment, the electrodes 200 are made from materials that have portions of the electrodes 200 that are not electrically conductive.

The drug 134 can be released into the vessel 104 upon timed, natural, electrical or otherwise activation, or into the vessel wall 103.

In variations of the device, an insulation layer between the nitinol substrate and the electrodes (e.g., platinum or gold) comprises a silicon dioxide or other insulation material. Alternatively, a layer of aluminum oxide can be provided to prevent the degradation and erosion of layers.

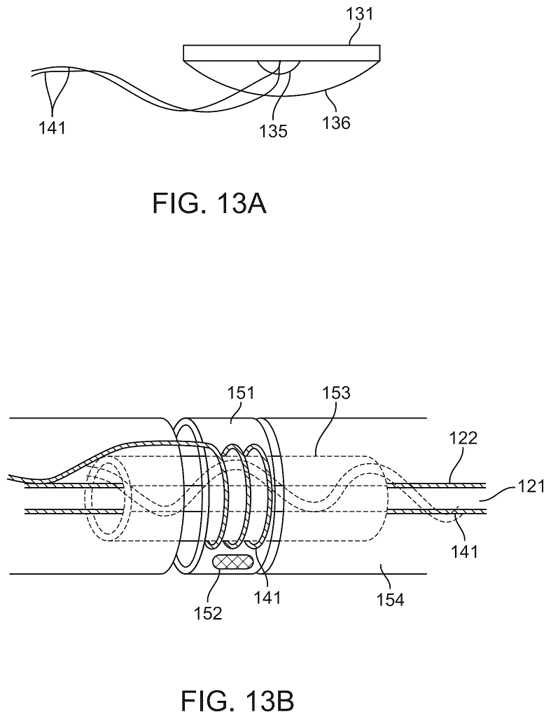

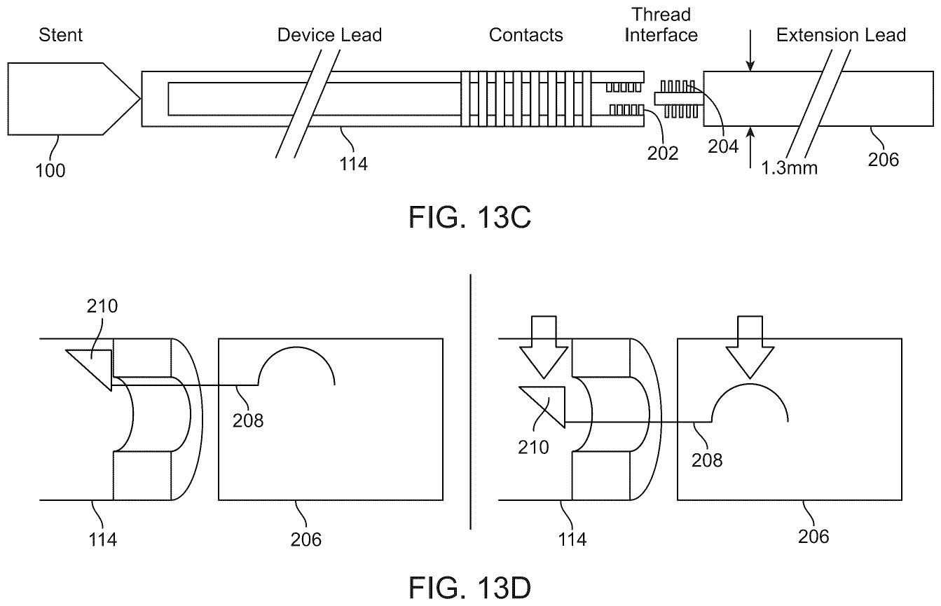

The electrode wires 141 can be electrically coupled to respective electrodes in the manner shown in FIG. 13A. As shown, the electrical attachment 135 and the back face of the electrode is covered in a non-conductive substance 136.

The lead wires 141 can be wrapped around the stent 101 and along a shaft 121.

As shown in FIG. 13B, the electrode lead wires 141 are wrapped around the shaft 121 and covered in insulation 122 forming a wire bundle or cable. A sleeve 153 wraps around the wire bundle at the location of the contact 151, whereby at least one wire 141 is wrapped around the sleeve 153 and connected to the contact 151 at a connection weld point 152. The over-molding 154 ensures a uniform diameter is present between contacts.

The sleeve 153 covers the wire bundle 142 with an exposed section of wire 141 attached 152 to a contact 151.

Distal electrodes and/or markers and/or buffers are also depicted 112 attached via a wire 114 to the stent 101. The shaft 121 is attached at the end of the stent at the attachment/detachment zone 115 and is shown passing through the sleeve 142 and electrode contacts 151 to exit behind past the connector securement point 155.

The lead wires 141 shown to be inside the sleeve 142 where they are wrapped around the shaft 121 where they make electrical contact at a contact weld 152 to the electrode contacts 151. An overcoat 154 is shown to ensure uniform diameter of the device between the contacts. The shaft 121 may be detached at the detachment zone 115 and removed following deployment in a vessel.

As shown in FIG. 13A, lead wires 141 are connected to electrode contacts 151. Electrode lead wires 141 are initially wrapped around a shaft 121 covered in insulation 122 forming a wire bundle or cable. A sleeve 153 is placed around the wire bundle at the location of the contact, whereby at least one wire 141 is wrapped around the sleeve and connected to the contact 151 at a connection weld point 152. Over-molding 154 may be used to ensure a uniform diameter is present between contacts.

As particularly shown in FIG. 5B, the stent shaft 121 is coated in an insulative layer 122, has a plurality of wires 141 that are insulated 143 and grouped in an insulated bundle 142 wrapped around it. A sleeve 153 covers the wire bundle 142 with an exposed section of wire 141 attached 152 to a contact 141.

The wires 141 are made from electrically conductive materials including but not limited to Platinum, Platinum/Tungsten, Stainless Steel, Nitinol, Platinum/Iridium, Nickel-Cobalt Alloys, or other conductive and biocompatible materials.

The wires 141 are between 10 um and 100 um thick (diameter), stranded cable or monofilament, and connect the electrodes 200 to the contacts 151. Alternatively, the wires 141 connect the electrode 200 to wireless circuitry retained on the stent or shaft.

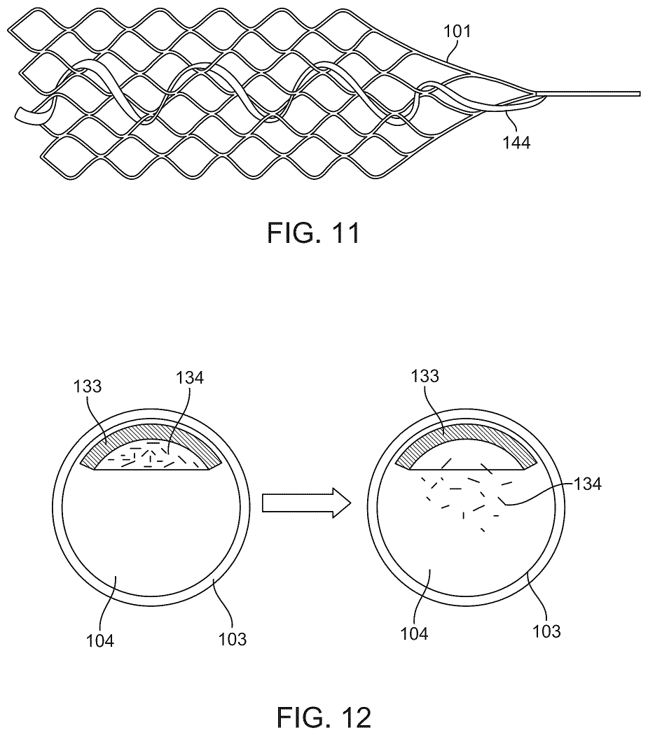

The wires 141 are insulated with non-conductive material (ie, Teflon or polyimide). The wires 141 are wrapped around the stent struts in a sinusoidal pattern as shown in FIG. 11. Alternatively, the wires 141 are wrapped in a helical tube or wire bundle or cable, with the wire or bundle between 300 um and 2 mm in diameter (thickness)

The wires 141 are connected to contacts 151 using wire wrapping, conductive epoxy, welding, or other electrically conductive adhesion or connection means.

FIG. 13C illustrates a variation of a lead 114 coupled to a device 100. In some circumstances, there may be a need retract the device for repositioning of the device after a sub optimal placement. FIG. 13C illustrates one variation of a lead 114 having a threaded screw terminal 202 that connects the existing lead to an extension lead 206. The lead can include a female threaded portion 202, which mates with a male portion 204 on the extension lead. Placement of the threaded portion on the interior of the lead reduces the risk that the male portion damages the spring contacts that the device 100 lead fits into after the extension lead has been removed.

FIG. 13D illustrates an alternative to the screw terminal design shown in FIG. 13C. In this variation, the lead 114 includes a locking mechanism 208. A variation of the locking mechanism can be based on pressure, where pressure on a selected portion of the lead would enable a latch 208 to open or close. Benefits of this would be to reduce the likelihood of any twisting of the device during delivery to detach the prematurely. In this variation, the latch 210 on the extension 206 locks into the lead 114. When an area on the extension is pushed (red arrows) the latch 210 releases from the lead 114 and can be either pushed into the lead (to attach) or be pulled from the lead (to release). Multiple latches would be placed around the circumference of the extension 114 lead, although one is shown here for purposes of illustration.

The control unit 12 can be a wireless controller, relaying information and power through the skin wirelessly.

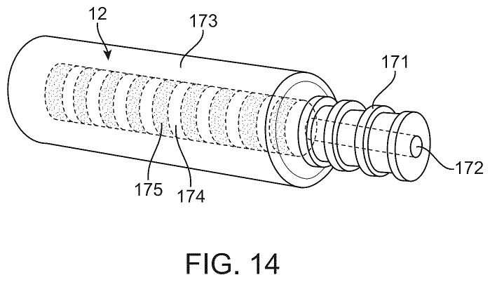

The connector block 12 in FIGS. 14-16 are passive devices (ie, no circuitry). Essentially, it functions as an intermediate connection between the device 100 and external equipment. The device 100 is inserted into the connector block 12 whereby the device 100 contacts make electrical contact with internal contacts contained within the connector block 12. These internal contacts of the connector block 12 then form a thicker wire bundle which passes through the skin (the rest of the connector block is implanted) and can be connected to external equipment.

The control unit 12 shown in FIG. 14 is shaped to receive and make electrical connection with the lead 14. The control unit include contacts rings mounted on the inside. Here, the connector block 12 is secured and ensured water-tight through attachment of silicone and/or sutures at the grooved end.

The wireless system that is implanted on the stent directly is essentially the same (although a miniaturised version) of the wireless system 12 in FIGS. 2A and 2B.

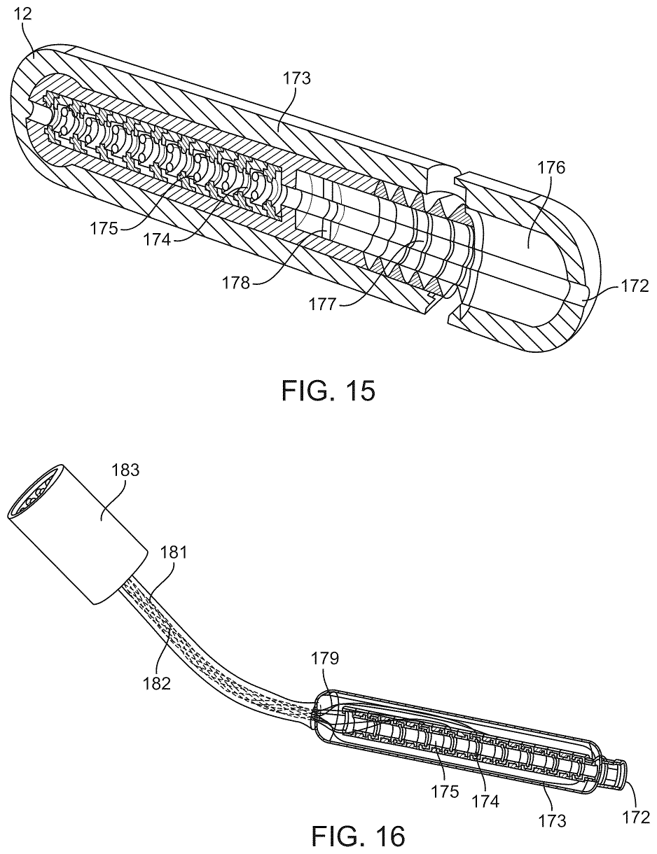

As shown in FIG. 15, the electrode lead 14 is inserted and a silicone gasket is used to make a watertight seal following

FIG. 16 depicts a connector block whereby the electrode lead 14 can be thread through the connection opening 172 whereby the contacts connect with the electrically conductive connectors 175 inside the connector block body 173. Separation and electrical insulation and water-tightness is increased through silicone (or otherwise) separators 174. Contacts 175 are welded (or otherwise) to connector block wires 179 that may form a silicone or otherwise 181 encased bundle 181 to terminate at a wireless or direct electrical connection port 183.

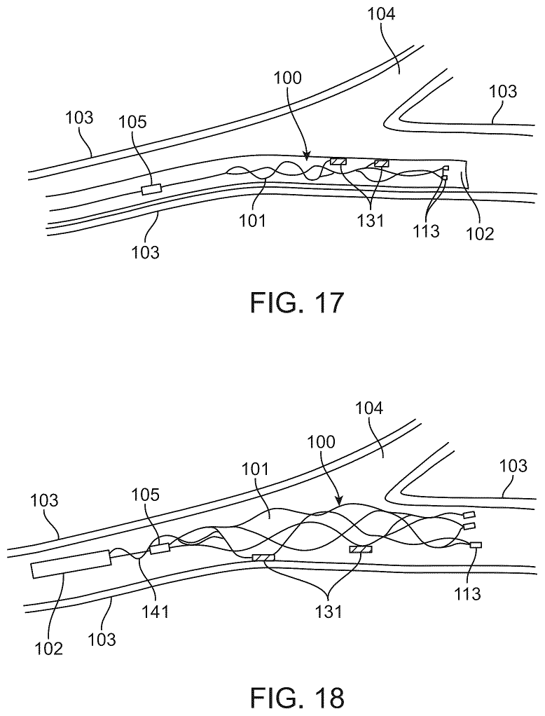

The device 100 can be movable between an insertion position shown in FIG. 17 and the deposition or scaffolding position shown in FIG. 18.

In the insertion position, the device 100 is contracted and thus thin enough to be threaded through the vasculature pathway from within a catheter from an entry point (ie, the jugular vein) to a deposition point (e.g., the motor cortex).

When arranged in the deposition or scaffolding position, the device 100 is in an expanded condition where scaffold electrodes mounted on the outside of the stent 101 as pressed against the vessel wall. This expanded position anchors the device 100 in its location within the vessel 103. Further, this deposition position is designed such that it has a minimal effect on blood flow integrity through the vessel 103 in which the device 100 is deposited. The scaffolding position may be synonymous to a spring, coil or helical strand, whereby the device 100 is in contact with the vessel wall only, reducing the effect on blood flow. Electrodes 200 may also be mounted on the inside of the stent 101 such that information from fluid flowing through the expanded stent 101 can be measured. For a stent 101 to be removed or relocated, additional shafts (other than that used for initial deployment) are required. These are explained in the context of this invention, with both single tapered and double tapered designs used.

To enable the device 100 to be arranged in multiple positions, the material used is such that multiple states are possible. These materials include, but are not limited to, Nitinol and other shape memory alloys and polymers. Further, to enhance the long term biocompatibility of the device 100, the polymers may be bioabsorbable or biodegradable, with a time of degradation similar to the time in which fibrosis occurs over the device 100. Hence, the electrodes 200 (which preferably are not designed to degrade, and may be made from Nitinol, shape memory alloys, conductive polymers, other non-shape memory alloys and inert and biocompatible metals such as platinum, iridium, stainless steel and gold) will be all that remains of the initial device 100 and will become embed inside the blood vessel 103, further enhancing the stability of the device 100 at the location of deposition.

FIG. 17 depicts a medical device 100 during implantation (surgical deployment phase) as it is being thread through vessels 104 inside a catheter 102. The stent 101, electrodes 200, stent detachment zone 105 and stent distal markers/electrodes/buffers 113 are shown, as are the vessel walls 103. Here, the catheter 102 is being used to select and direct the device into the desired vessel 104.

FIG. 18 depicts a medical device 100 in the expanded or deposition or scaffolding position comprising a stent 101, distal olives and/or proximity markers 113, a plurality of electrodes 200, lead wires 141 and a stent detachment zone 105 being deployed in a blood vessel 104 through a deposition catheter 102. Stent 101 mounted electrodes 200 are in direct apposition with the vessel wall 103 and are depicted as not interruptive of blood flow to any vessel (both the vessel the device is deployed in and other connected vessels).

The platinum C-shaped ground electrode 167 shown in FIG. 25 is embedded in silicone 181 with a red helical lead wire 141 that is attached to a standard electrical terminal 169. Dacron mesh is used to assist secure the electrode and wire to tissue.

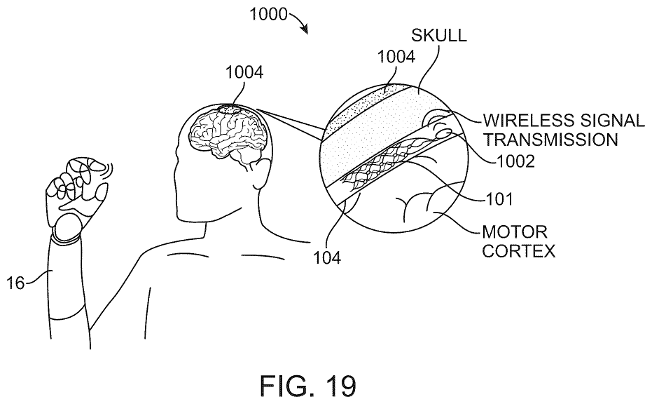

FIG. 19 shows a wireless electrode system 1000 showing electrodes mounted on a stent 101 within a blood vessel 104 overlying the motor cortex in a human that are picking up neural information and relaying this information to a wireless transmitter 1002 located on the stent 101. Note the stent 101 has been deployed and the stylet has been removed (ie, only the stent 101, electrodes, electrode wires and wireless system 1002 remains). The information is wirelessly transmitted through the skull to a wireless received 1004 placed on the head, which in turn, decodes and transmits the acquired neural information to a prosthetic limb 16.

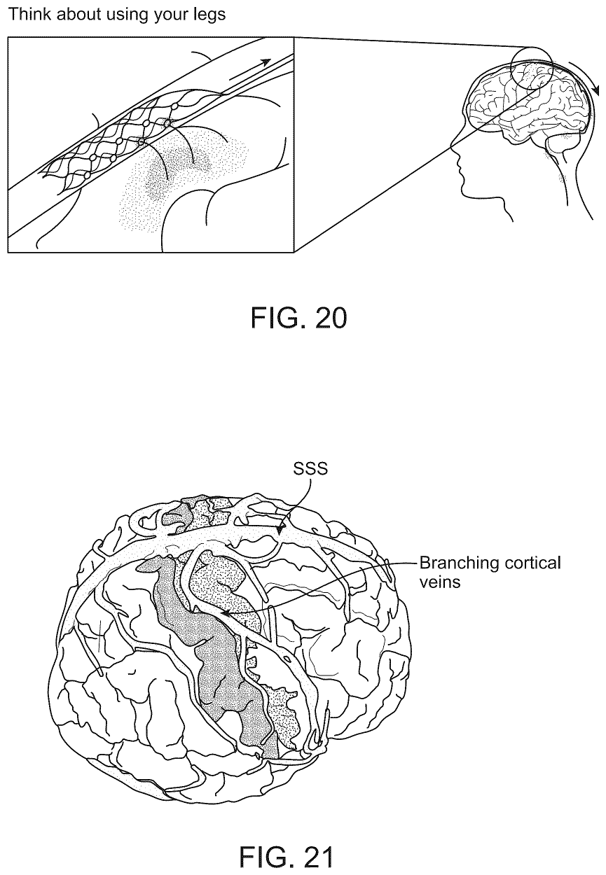

As shown in FIG. 20, the device 100 can be used to record neural information or stimulation of neurons from the superior sagittal sinus (SSS) or branching cortical veins of a patient using the device 100, including the steps of: (a) implanting the device in either the superior sagittal sinus or branching cortical veins; (b) receiving activity; and (c) generating data representing said activity; and (d) transmitting said data to a control unit. Stent 101 implanted in SSS over motor cortex acquiring (i.e. receives) signals that are fed through the wire to external equipment 12.

FIG. 21 shows an image reconstruction of a human brain (eyes facing left) demonstrating superior sagittal sinus and branching cortical veins near the motor cortex (red) and sensory cortex (yellow)

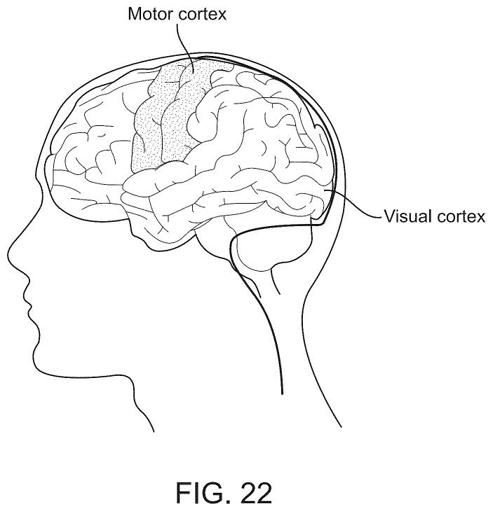

FIG. 22 shows a method of for stimulation and recording neural information or stimulation of neurons from the visual cortex of a patient using the device 100, including the steps of: (a) implanting the device in a vessel in the visual cortex of the patient; and (b) recording neural information associated with the vessel or stimulating neurons in accordance with received stimulation data.

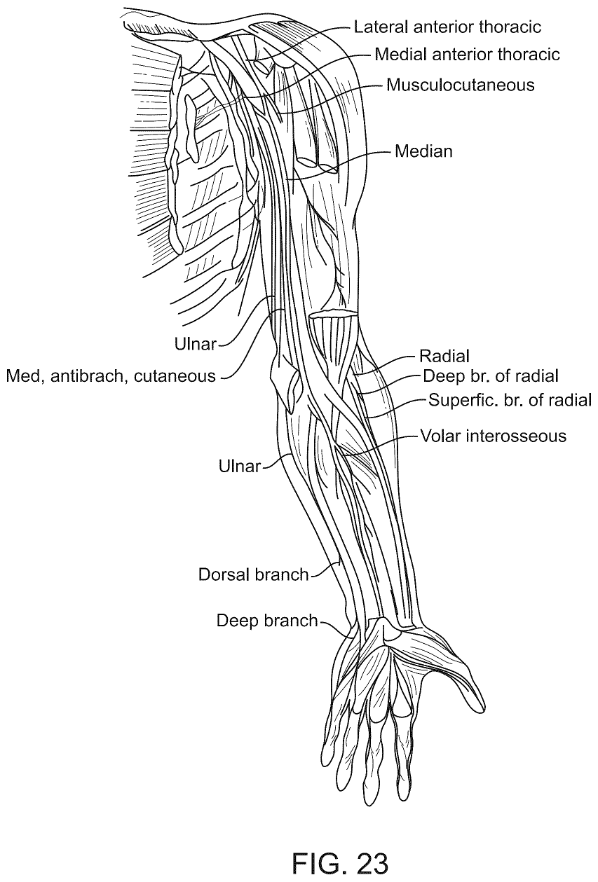

As particularly shown in FIG. 23, the device 100 is delivered through a vessel 104 deposited in a muscle for direct muscular stimulation or recording.

The device 100 can be delivered through a vessel adjacent to a peripheral nerve (such as shown in FIG. 23) for stimulation or recording.

The device is delivered through a vessel adjacent to a sympathetic or parasympathetic nerve for stimulation.

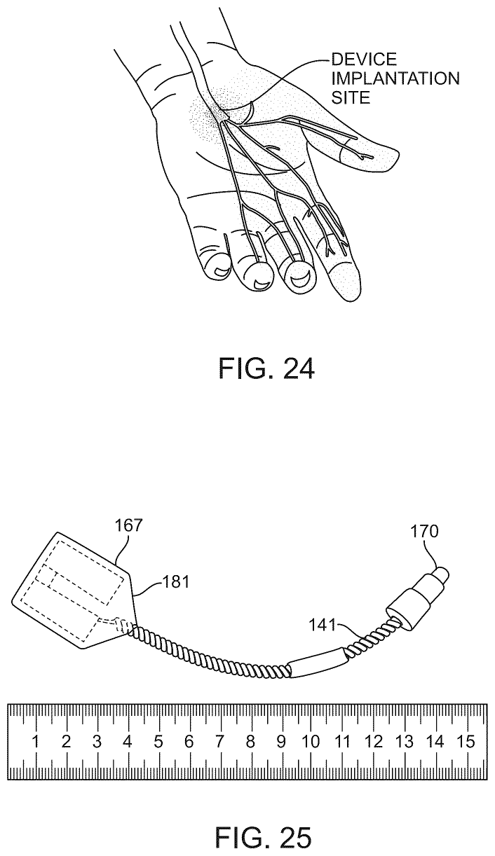

As shown in FIG. 24, one example of a peripheral nerve (the median nerve in this example) showing possible implant location to enable neural stimulation or measurement.

Placement of the electrodes in a specific pattern (e.g., a corkscrew configuration or a configuration of three linear (or corkscrew oriented) lines that are oriented 120 degrees from each other) can ensure a deployed electrode orientation that directs electrodes towards the brain. Once implanted, orientation is not possible surgically (i.e., the device will be implanted and will be difficult if not impossible to rotate). Therefore, variations of the device will be desirable to have an electrode pattern that will face towards the desired regions of the brain upon delivery.

Electrode sizing should be of a sufficient size to ensure high quality recordings and give large enough charge injection limits (the amount of current that can be passed through the electrodes during stimulation without damaging the electrodes which in turn may damage tissue). The size should also be sufficient to allow delivery via a catheter system.

As discussed above, embedding the electrode and conductive path presents advantages in the mechanical performance of the device. Furthermore, embedding of electrodes provides the ability to increase the number of electrodes mounted on the structure give that the conductive paths (30-50 μm×200-500 nm) can be smaller than traditional electrode wires (50-100 μm).

Manufacture of thin-film stents can be performed by depositing Nitinol or other superelastic and shape memory materials (or other materials for deposition of electrodes and contacts (including but not limited to gold, platinum, iridium oxide) through magnetron sputtering in a specific pattern (56) using a sacrificial layer (58) as a preliminary support structure. Removal of the support structure (54) enables the thin film to be further structured using UV-lithography and structures can be designed with thicknesses corresponding with radial force required to secure the electrodes against a vessel wall.

Electrical insulation of electrodes is achieved by RF sputtering and deposition of a non-conductive layer (52) (eg, SiO) onto the thin-film structure (54). Electrodes and electrode tracks (50) are sputter deposited onto the non-conductive layer (using conductive and biomedically acceptable materials including gold, Pt, Ti, NiTi, PtIr), with an additional non-conductive layer deposited over the conductive track for further electrical isolation and insulation. As shown, conducting path 50 is left exposed to form the electrode 138 (similarly, a contact pad area can remain exposed). Finally, the sacrificial layer 56 and substrate are removed leaving the stent structure 101.

In certain variations where the base structure 54 comprises superelastic and shape-memory materials (i.e. Nitinol), the stent structure 101 can be annealed in a high vacuum chamber to avoid oxidation during the annealing process. During heat treatment, the amorphous Nitinol structure 54 crystallizes to obtain superelasticity and can be simultaneously shape set into a cylindrical or other shape as desired. The structure 101 can then be heat treated.

In other variations, the electrodes can be manufactured using 2.5-dimensional structuring or alternative materials to enhance the surface area of the electrodes, resulting in lowering impedance, increasing charge injection capacity, and enhanced stimulation and recording capabilities. In some variations, platinum black or iridium oxide can be used as the material for the electrode, or surface coated onto the electrode, as it can provide a higher microscopic surface area without increasing the geometric footprint of the electrode. In other variations, the enhancement of surface area can be accomplished by electrochemical roughening or laser ablation of the electrode to create pits in the electrode surface increasing effective surface area.

In other variations, different geometric patterns can be structured onto the electrodes to increase surface area, such as by photolithography or etching. For example, some structured patterns on the electrode can include pillars or trenches forming a cross-hatch, an “S-shaped” pattern, horizontal and/or vertical lines, or circular patterns or grooves.

Systems described herein can be used to enhance cognitive output, for improvements in such areas as: learning, memory, training, motor tasks, etc. Transcranial Direct-Current Stimulation (TDCS) and Transcranial Magnetic Stimulation (TMS) have been shown to have potential applications in improved attention, learning, and motor outputs. Implantation of an intravascular stimulation device into the appropriate area could potentially create more reliable, long term improvements to cognitive outputs using less energy due to increased access and proximity to the regions of interest.

The systems and implants described herein can record and process neural activity to control devices that are internal and/or external to a user's body via a brain machine interface. Such processing can be done with one or more processors or microprocessors that are, for example, integrated or otherwise in communication with one or more stent devices 100 and/or with the telemetry unit 12. The processors can be programmed with or be capable of calling a variety of control algorithms to process the neural signals received from the brain and/or from elsewhere in the body. This includes neural signals received from both the sympathetic and/or parasympathetic pathways. For example, an electrode array (e.g., the stent based electrode array 100) can sense cortical and/or sub-cortical neural activity, and can relay such activity to a processor to control a brain machine interface. The brain machine interface can be linked to internal and/or external devices. Cortical and subcortical locations can include, for example, the primary motor cortex (M1), the supplementary motor area (SMA), the posterior parietal cortex (PPC), the primary somatosensory cortex (S1), the cerebellum, the thalamus and the brain stem. Neural activity in areas outside of the brain can also be sensed and processed, for example, from the spinal cord, muscles and organs such as the heart, lungs, stomach, kidneys and pancreas. The control algorithms can process neural activities that are sensed or recorded by the system to generate control signals. The generated control signals can allow for the neural control of one or multiple external devices, internal devices, parts of the body, or any combination thereof. For example, the algorithms can produce control signals that actuate some part of a device and/or that stimulate tissue.

The algorithms can process sensed neural activity from one or more neural areas to determine, for example, whether a user intends to act, and if so how much. If the sensed neural signals correspond to intended action, the brain machine interface can generate control signals that actuate a device associated with the action intended. For example, where a user has a prosthetic arm linked to a brain machine interface, the user can think about raising their arm and the system can detect this intent by processing the neural signals that are associated with this action. The system can transform this detected intent into a control signal to raise the prosthetic arm according to the user's intended action.

Various algorithms can be used to decode or otherwise determine a user's intent as well as determine whether the sensed or decoded intent corresponds to a user's intended action. For example, the system can sense signals from multiple brain areas to rely on and detect natural synergies that exist between multiple brain areas when a user mentally forms an intent (e.g., a motor intent). Such an intent determination algorithm relies on the fact that any given intent will be replicated in multiple areas of the brain and be supplemented with additional information. For example, the system can detect and use natural cortical and/or subcortical synergies for informing the outputs of the brain machine interface when determining intent. In such a case, the system can determine a user's intent by processing neural signals from two or more neural areas and then making a determination of whether the two or more sets of neural signals are associated with one another before generating an output signal. Sensing and analysing neural synergies can reduce the risk of accidentally activating devices in communication with the brain machine interface since such a decoding process relies on multiple areas of the brain as opposed to just one, and takes advantage of the neural redundancies or lack thereof that naturally result. Utilizing such synergies can therefore enable for more accurate and reliable identification of a person's intent. This can in turn allow for the generation of more accurate and reliable control signals, as well as instill greater confidence in users for the device. The system can also determine a user's intent without relying on neural synergies, for example, by processing neural signals from a single area without associating or comparing the signals to signals from other neural areas.

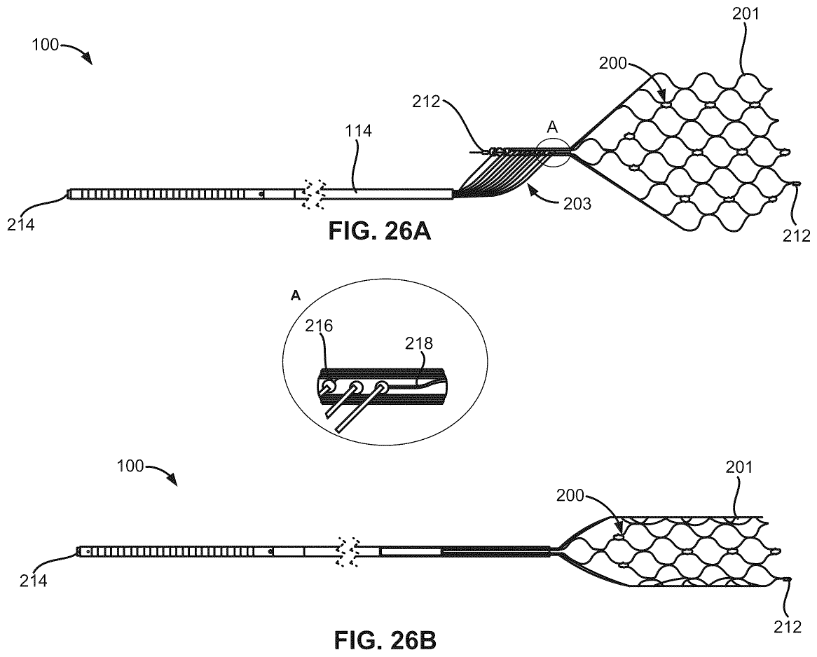

FIGS. 26A and 26B illustrate one variation of the device 100 having an electrode array 210 with one or more electrodes 200 spaced apart from each other and connected to a lead 114.

The device 100 can comprise one or more radiopaque markers 212. The radiopaque markers can be found on the lead 114 or the electrode array 201. As seen in detail A, an epoxy 216 can be applied to the radiopaque marker 212 such that the marker is coupled to the lead.

The proximal end of the device can comprise a threaded tip 214. The threaded tip 214 can be connected to a delivery catheter (not shown).

The device 100 can have a lead 114 which houses one or more electrical vias 203. As described previously herein, each of the electrical vias 203 can be coupled to each electrode 200. As seen in detail A, the vias 203 can be attached to an inner surface of lead 114 via a conductive epoxy 216. The vias 203 can also be fixed within the lead 114 to each other by an adhesive 218.

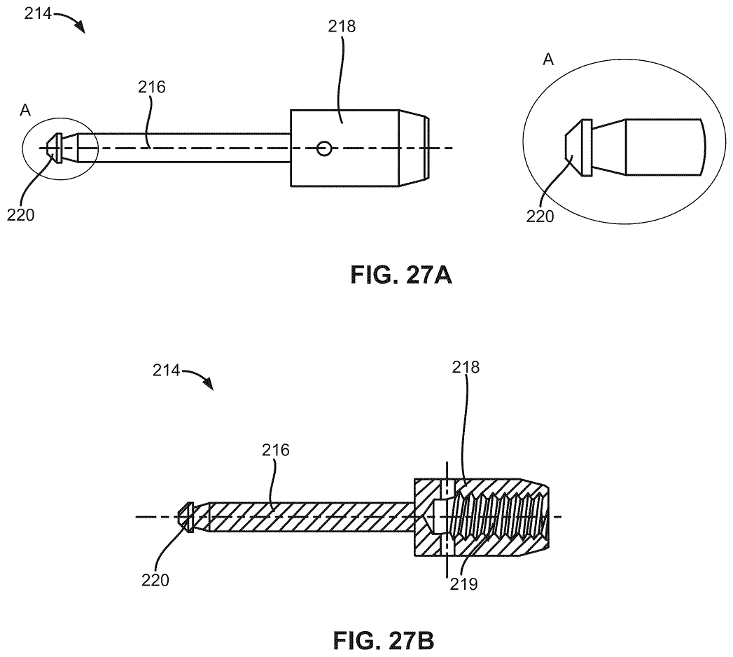

FIGS. 27A and 27B illustrate the threaded tip 214 according to one variation of the invention. Threaded tip 214 can comprise a shaft portion 216, a head portion 218 at a distal end, and a bulb portion 220 (see detail A) at a proximal end. The head portion 218 can comprise a female threaded portion 219 within. The head portion 218 can comprise an outer diameter of about 0.04 inches to about 0.06 inches (e.g., about 0.05 inches) and an inner diameter of about 0.02 inches to about 0.03 inches (e.g., about 0.024 inches). The head portion 218 can be substantially parallel to a longitudinal axis of the threaded tip 214 and can taper at an angle of about 30 degrees.

The threaded tip 214 can be inserted into a proximal connector of the lead 114 at the proximal end. To this end, bulb portion 220 can be melted into the proximal end of the lead 114 and can help mechanically retain the threaded tip 214 to the lead body.

As seen in detail A, the bulb portion can comprise a diameter of about 0.013 inches which tapers down to about 0.008 inches at the proximal most end.

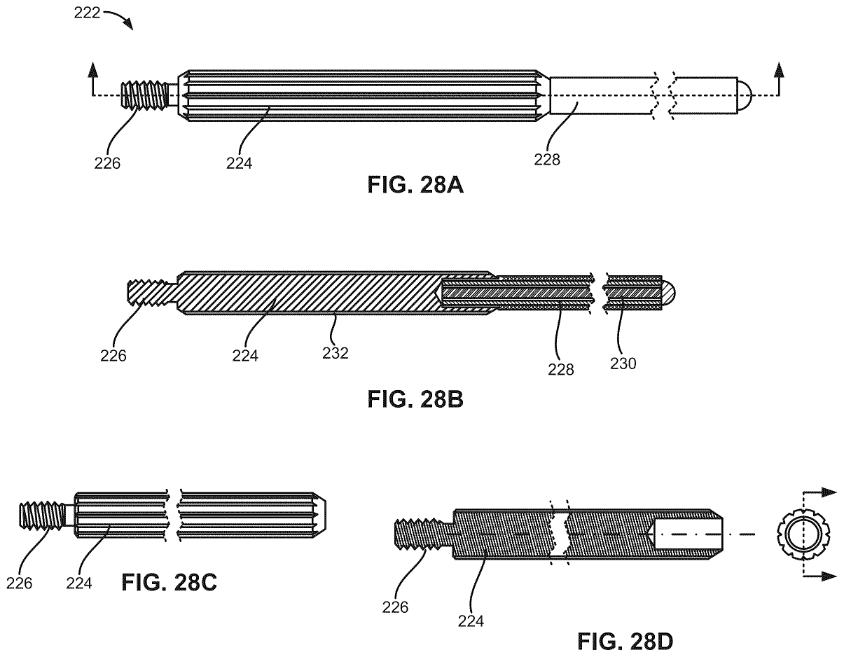

FIGS. 28A to 28D illustrate a lead extender 222 according to one variation of the invention. The lead extender 222 can comprise a shaft 224, a male threaded portion 226 for coupling with the threaded portion 219 of threaded tip 214, and a lead extender tube 228. The lead extender 222 can comprise a diameter of about 0.05 inches.

As seen in FIG. 28B, the cross-sectional view of FIG. 28A, the lead extender tube 228 can also comprise a core wire 230 within to provide stability to the lead extender tube 228. A connector 232 can be disposed around the shaft 224 and can couple the shaft 224 to the lead extender tube 228.

As seen in the cross-sectional view of FIG. 28D, the shaft 224 can comprise an unraised knurl along the outer diameter.

The lead extender tube 228 can comprise a PEBAX outer layer to heat shrink, enabling the lead extender tube 228 to seal against a silicone valve on a rotating hemostatsis valve during flushing of the delivery catheter. Without the PEBAX outer layer, the silicone valve would not be able to seal and saline would backflow during catheter flushing.

Many modifications will be apparent to those skilled in the art without departing from the scope of the present invention.

The reference to any prior art in this specification is not, and should not be taken as, an acknowledgment or any form of suggestion that the prior art forms part of the common general knowledge in Australia.

In this specification and the claims that follow, unless stated otherwise, the word “comprise” and its variations, such as “comprises” and “comprising”, imply the inclusion of a stated integer, step, or group of integers or steps, but not the exclusion of any other integer or step or group of integers or steps.

References in this specification to any prior publication, information derived from any said prior publication, or any known matter are not and should not be taken as an acknowledgement, admission or suggestion that said prior publication, or any information derived from this prior publication or known matter forms part of the common general knowledge in the field of endeavor to which the specification relates.

Any elements described herein as singular can be pluralized (i.e., anything described as “one” can be more than one). Like reference numerals in the drawings indicate identical or functionally similar features/elements. Any species element of a genus element can have the characteristics or elements of any other species element of that genus. Some elements may be absent from individual Figs. for reasons of illustrative clarity. The above-described configurations, elements or complete assemblies and methods and their elements for carrying out the disclosure, and variations of aspects of the disclosure can be combined and modified with each other in any combination. All dimensions shown in the drawings are exemplary.

We claim:

1. A method of sensing electrical activity from one or more neural regions of a subject, the method comprising:
advancing a device to or through a blood vessel in the subject, where the device comprises an electrode array having a plurality of electrodes;
sensing electrical activity via the plurality of electrodes;
detecting an electrical signal of a subject, where the plurality of electrodes are spaced apart on the electrode array such that the plurality of electrodes are each configured to record distinct electrical signals from each of the one or more neural regions, wherein the plurality of electrodes are spaced apart such that a resulting coverage field of each of the plurality of electrodes minimizes overlap between each of the plurality of electrodes; and
analysing the distinct electrical signals using a control unit.

2. The method according to claim 1, further comprising deploying the electrode array after advancing the device to or through the blood vessel, where deployment of the electrode array brings each of the plurality of electrodes into engagement with a wall of the blood vessel.

3. The method according to claim 1, wherein the device is an intravascular device deployed within the blood vessel.

4. The method according to claim 1, wherein the device is a transvascular device.

5. The method according to claim 4, wherein deploying the electrode array further comprises deploying the electrode array to the subarachnoid space.

6. The method according to claim 4, wherein deploying the electrode array further comprises deploying the electrode array to the cortical surface.

7. The method according to claim 4, wherein deploying the electrode array further comprises deploying the electrode array within the cortical tissue.

8. The method according to claim 1, wherein the plurality of electrodes record distinct electrical signals from one or more neural regions at the spinal cord of the subject.

9. The method according to claim 8, wherein the device is an intravascular device deployed within a blood vessel of the spinal cord.

10. The method according to claim 8, wherein the device is a transvascular device.

11. The method according to claim 10, wherein deploying the electrode array further comprises deploying the electrode array to the epidural space.

12. The method according to claim 10, wherein deploying the electrode array further comprises deploying the electrode array to the subarachnoid space.

13. The method according to claim 10, wherein deploying the electrode array further comprises deploying the electrode array to the spinal cord surface.

14. The method according to claim 10, wherein deploying the electrode array further comprises deploying the electrode array within the spinal cord.

15. The method according to claim 1, further comprising extracting the distinct electrical signals via the control unit and distilling information from the distinct electrical signals to generate one or more control signals sending specific instructions to the device.

16. The method according to claim 15, wherein extracting the distinct electrical signals comprises temporally filtering the distinct electrical signals.

17. The method according to claim 15, wherein extracting the distinct electrical signals comprises spatially filtering the distinct electrical signals.

18. The method according to claim 15, wherein extracting the distinct electrical signals comprises thresholding the distinct electrical signals.

19. The method according to claim 1, wherein two or more of the plurality of electrodes are configured to record distinct electrical signals from each of the one or more neural regions.

20. A method of sensing electrical activity from one or more neural regions, the method comprising:
advancing a device through the subarachnoid space in a subject, where the device comprises an electrode array having a plurality of electrodes;
detecting an electrical signal of a subject, where the plurality of electrodes are spaced apart on the electrode array such that the plurality of electrodes are each configured to record distinct electrical signals from each of the one or more neural regions; and
analysing the distinct electrical signals using a control unit, wherein the distinct electrical signals each correspond to distinct recordings of electrical activity within the subject.

21. The method of claim 20, further comprising deploying the electrode array within the subarachnoid space adjacent to a surface of the brain, wherein deployment of the electrode array brings each of the plurality of electrodes into engagement with the surface of the brain.

22. The method of claim 20, wherein the plurality of electrodes comprise one or more pairs of electrodes, wherein each pair of electrodes is configured to record distinct electrical signals from each of the one or more neural regions.

23. The method of claim 20, wherein the plurality of electrodes comprise one or more sets of three electrodes, wherein each set of three electrodes is configured to record distinct electrical signals from each of the one or more neural regions.

24. The method of claim 20, wherein the plurality of electrodes comprise one or more sets of two or more electrodes, wherein each set of two or more electrodes is configured to record distinct electrical signals from each of the one or more neural regions.

25. The method according to claim 20, further comprising extracting the distinct electrical signals via the control unit and distilling information from the distinct electrical signals to generate one or more control signals sending specific instructions to the device.

26. The method according to claim 20, wherein extracting the distinct electrical signals comprises temporally filtering the distinct electrical signals.

27. The method according to claim 20, wherein extracting the distinct electrical signals comprises spatially filtering the distinct electrical signals.

28. The method according to claim 20, wherein extracting the distinct electrical signals comprises thresholding the distinct electrical signals.

29. The method according to claim 20, wherein two or more of the plurality of electrodes are configured to record distinct electrical signals from each of the one or more neural regions.

30. A method of providing stimulation to one or more neural regions of a subject, the method comprising:
advancing a device to or through a blood vessel in the subject, where the device comprises an electrode array having a plurality of electrodes;
stimulating the one or more neural regions via the plurality of electrodes;
where the plurality of electrodes are spaced apart on the electrode array such that the plurality of electrodes are each configured to stimulate one or more distinct neural regions, wherein the plurality of electrodes are spaced apart such that a resulting stimulation field of each of the plurality of electrodes minimizes overlap between each of the plurality of electrodes, wherein stimulation is delivered using a control unit.

31. A system for sensing electrical activity from one or more neural regions of a subject, the system comprising:
an electrode array comprising a plurality of struts, where the electrode array is configured to transition between a constrained configuration and a deployed configuration in which the electrode array is implanted within a blood vessel;
a plurality of electrodes spaced apart on the electrode array such that the plurality of electrodes are each configured to record distinct electrical signals from each of the one or more neural regions, wherein the plurality of electrodes are spaced along a longitudinal axis of the electrode array, wherein the plurality of electrodes are each configured to sense distinct electrical activity from distinct neural regions within the blood vessel; and
a control unit configured to receive and analyze the distinct electrical signals, wherein the distinct electrical signals each correspond to distinct recordings of electrical activity within the subject.

32. The system according to claim 31, wherein the plurality of electrodes are offset from each other in a plane transverse to the longitudinal axis of the electrode array.

33. The system according to claim 31, wherein a diameter of the electrode array is 10.5 mm.

34. The system according to claim 31, wherein the electrode array comprises a cylindrical shape in the deployed configuration.

35. The system according to claim 31, wherein the plurality of electrodes are spaced in rows of electrodes.

36. The system according to claim 31, further comprising a plurality of electrical vias each connected to each of the plurality of electrodes, wherein the plurality of electrical vias is coupled to a proximal end of the electrode array and connects to a lead and a control unit of the system.

37. The system according to claim 36, wherein each of the plurality of electrical vias are routed so as to not cross another electrical via.

38. The system according to claim 31, wherein the plurality of electrodes comprise a substance configured to be released by the plurality of electrodes.

39. The system according to claim 38, wherein the substance is a therapeutic drug.

40. The system according to claim 38, wherein the substance is an anti-thrombogenic drug, and materials.
